(19) United States
(12) Patent Application Publication
(10) Pub. No.: US 2007/0141016 A1
(43) Pub. Date: Jun. 21, 2007

(54) The Shaving Stick

(75) Inventor: Kevin Monte de Ramos, Montreal, QC, NY (CA)

Correspondence Address: Kevin Monte de Ramos, 105-454 Rue de La Gauchetiere Ouest, Montreal, QC, H2Z-1E3, CA

(73) Assignee: Kevin Monte de Ramos, Montreal, QC (CA)

(21) Appl. No.: 11/306,176

(22) Filed: Dec. 19, 2005

(51) Int. Cl. A61K 8/36; B26B 19/00

(52) U.S. Cl. 424/73; 30/34.05

(57) ABSTRACT

The shaving stick is a means of dispensing shaving lubricant directly onto the skin without the need of other tools. The saving stick is the combination of a shaving lubricant and an applicator assembly; whereby, the shaving lubricant may be any formulation of shaving soap, cream, gel, lotion, or liquid. As such, users of our invention, the shaving stick, may apply the shaving aid directly to the face, legs, under arms, etc. without the use their hands, as is needed with shaving creams, astringents, and other lubricants on the market. Current embodiments of our invention resemble visually and functionally the various underarm deodorants available in the market today. By replacing the deodorant with a shaving lubricant, the shaving stick overcomes the many inconveniences associated with current shaving aids.

### FIELD OF INVENTION

The present invention relates to the delivery of shaving aids directly to the skin of men and women wishing to use either a manual razor or electric shaver.

### BACKGROUND

Shaving has undoubtedly been with us since the beginning of human civilization. While technology has improved over the centuries, the overall process of shaving facial and body hair has changed little. A sharp blade is passed over the skin, severing hair at the skin's surface.

The distress experienced by men and women during shaving has led to the development of aids; namely astringents, soaps, gels, creams, and lotions. The shaving aids on the market today adequately relieve skin irritations, but fail to address other inconveniences associated with the use of these products.

Shaving aids available in the market today come in three primary forms: (a) soaps whipped into a lather and then applied to the face using a brush, (b) creams/gels/lotions dispensed from the container into the hand and then spread across the area to be shaved, or as a (c) liquid astringent splashed onto the body.

The use of these shaving aids have a single unifying inconvenience: each requires an intermediary tool to apply the shaving aid onto the skin; namely the user's hand. Additionally, shaving creams/gels/lotions accumulate to hide the hairs to be shaved. As such, it is often necessary to manually remove excess lubricant before passing the blade near critical hair lines; a man's sideburn and mustache or a women's bikini line. Another inconvenience is that shaving kits, cans of shaving cream, and bottles of shaving astringents take up limited space in our travel bags.

The only prior art found to specifically address the inconvenience of existing shaving aids is that of Dana K. Griffith in December of 2005 (U.S. Pat. No. 6,893,631). To avoid the mess of dispensing shaving cream from a can into the hand, Dana Griffith offers society a hollow shaving lotion packet containing a towelette impregnated with a shaving lotion of a specific composition and a method of preparing skin and unwanted hair for shaving.

### SUMMARY OF THE INVENTION

Our invention, the shaving stick, allows men and women to apply a shaving aid directly to the skin. Our innovation was inspired by the multitude of deodorant sticks available on the market. While shopping, I asked, why not mold shaving soaps to fit the deodorant container?

Returning home, I did just that. The outcome was a product that appealed to both myself and my fiancee. The shaving stick has been used with both manual razors and electric shavers. We now use the shaving stick, both at home and on the road, because three important advantages overwhelm the prior art.

First, no intermediary tool is needed to apply the shaving aid onto the skin. Users are able to slide the shaving stick across the face; thereby, lubricating both the hair and the skin in preparation for shaving. Similarly, the shaving stick can be used under arm, on the leg, around the bikini line, etc. As a result, users of this invention can prepare for shaving without either hand contacting the shaving aid. This is a desirable enhancement over shaving creams, lathers, and lotions that typically applied with the user's hand.

Second, the shaving aid does not accumulate to hide the hairs targeted for shaving. Therefore, excess shaving creams/lotions/gels need not be removed before defining critical hair lines; such as, a man's sideburns and mustache or a women's bikini line. Because the shaving stick leaves behind only a thin film of lubricant per stroke, users of this invention can apply the appropriate amount of shaving lubricant; thereby, allowing them to monitor the area being shaved without difficulty.

Thirdly, the shaving stick requires no ancillary items to support its use. The shaving stick is a self-contained shaving aid. Additionally, shaving aids formulated as solids can last longer and take up less space than shaving aids formulated as liquids, gels, or creams. As a result, the shaving stick can be designed to address the needs of modern living by minimizing the size of the applicator assembly and the shaving lubricant.

Our invention differs significantly from that of Dana Griffith (U.S. Pat. No. 6,893,631) in that our innovation: (a) does not specify a formulation of shaving lubricant, (b) is not a moist towelette, and (c) can be applied without the users hand coming into contact with the shaving lubricant. As such, our invention can not be confused with prior art.

### BRIEF DESCRIPTIONS OF THE DRAWINGS

The invention is better understood when consideration is given to following descriptions and embodiments. The description references the annexed drawings wherein:

FIG. 1 is a front perspective view of the preferred embodiment of the shaving stick; and

FIG. 2 is a front perspective view of an alternate embodiment of the shaving stick, designed to prepare the armpit for shaving.

The same reference numerals refer to the same parts throughout both figures.

### DETAILED DESCRIPTION OF THE DRAWINGS

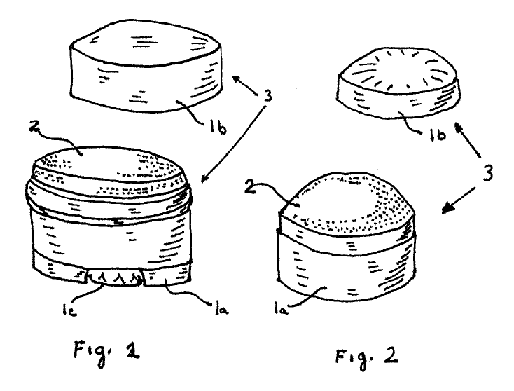

Referring now to the drawings, and particularly to FIGS. 1-2, a current embodiment of the present invention, the shaving stick, is shown and generally designated by the reference numeral 3.

In FIG. 1, the preferred embodiment of the shaving stick 3 is shown. The shaving lubricant 2 in this embodiment of the invention consists of commercial shaving soap molded to fit within the applicator assembly body 1a and its cover 1b. The shaving lubricant 2 can be raised by turning a dial 1c on the applicator assembly body 1a; thereby exposing the desired amount of shaving lubricant 2. To apply the shaving lubricant 2, users would wet the area to be shaved, hold the applicator assembly 1a in hand with the applicator assembly cover 1b removed, and move the shaving stick 3 across the area to be shaved until the hair and skin are adequately prepared for shaving.

In FIG. 2, an alternate embodiment of the shaving stick 3 is shown. The shaving lubricant 2 of this embodiment of the shaving stick 3 consists of shaving soap molded to fit atop the applicator assembly body 1a and beneath its cover 1b. In this embodiment of the shaving stick 3, the shaving lubricant 2 is shaped to facilitate the preparation of the armpits for shaving. This embodiment of the shaving stick 3 is used in the shower by persons wishing to shave their underarm hair. The applicator assembly cover 1b is removed to expose the shaving lubricant 2. The applicator assembly body 1a is held in hand and the shaving lubricant 2 applied via a back and forth motion; thereby preparing the armpits for shaving.

FIGS. 1-2 show only two embodiments of the shaving stick 3. The forgoing illustrations show only the principal of the invention. It is understood that other formulations of shaving lubricant 2, gels in particular, may fit entirely within the applicator assembly 1a-1c. It also understood that the applicator assembly can take many shapes and may be constructed to meet evolving consumer demand.

We fully expect those skilled in the art to modify the current embodiment of our invention to develop retail market niches. This fact is evidenced by the vast array of designs for underarm deodorant sticks on store shelves. It is undesirable and unintended to limit the invention to the exact construction and operation as shown or described. All suitable modifications and equivalents that combine a shaving lubricant with an applicator assembly shall be considered within the scope of this invention.

What is claimed is:

1. a means of dispensing shaving lubricant directly onto the skin without the need of other tools, comprising:
an applicator assembly; and
a shaving lubricant;
wherein, the combination represents the shaving stick.

2. The shaving lubricant of claim 1, wherein the shaving lubricant is any formulation of shaving soap, cream, gel, lotion, or liquid.

3. The applicator assembly of claim 1, wherein the shaving lubricant of claim 2 is contained by and dispensed from the applicator assembly directly onto the shaving area.
